(19) United States
(12) Patent Application Publication
(10) Pub. No.: US 2010/0049256 A1
(43) Pub. Date: Feb. 25, 2010

(54) ANTERIOR CERIVCAL PLATING SYSTEM

(76) Inventors: Dong Myung Jeon, Draper, UT (US); Patrick Moore, West Jordan, UT (US); Sang K. Lee, Seoul (KR)

Correspondence Address: MORRISS OBRYANT COMPAGNI, P.C., 734 EAST 200 SOUTH, SALT LAKE CITY, UT, 84102, US

(21) Appl. No.: 12/524,761

(22) PCT Filed: Jan. 30, 2008
(86) PCT No.: PCT/US08/01188

§ 371 Date: Oct. 20, 2009

Related U.S. Application Data
(60) Provisional application No. 60/887,213, filed on Jan. 30, 2007.

(51) Int. Cl. A61B 17/80

(52) U.S. Cl. 606/289; 606/286

(57) ABSTRACT

Anterior cervical plating systems including an elongated anterior cervical plate with bone screw bores disposed near two opposite ends. The anterior cervical plate incorporates a curved plane transverse to the longitudinal axis to conform to the anterior surface of a vertebra and is also curved along the longitudinal axis to conform to the lordotic curvature between the vertebrae and may feature at least one through window in a middle bridge portion to provide an interface for a drill and tap guide assembly. Paired locking mechanisms provided on the anterior cervical plate provide rigid fixation of the bone screws to the anterior cervical plate with the heads of the bone screws flush or recessed below the upper curved surface of the anterior cervical plate.

### CROSS-REFERENCE TO RELATED APPLICATION

This application claims the benefit of U.S. Provisional Application No. 60/887,213, filed 30 Jan. 2007, the disclosure of which is incorporated herein by reference in its entirety.

### TECHNICAL FIELD

The present invention concerns spinal instrumentation systems, such as for use with the cervical vertebrae. More particularly, the invention pertains to a plating system for use in treatment of the cervical spine.

### BACKGROUND

For a number of known reasons, bone fixation devices are useful for promoting proper healing of injured or damaged vertebral bone segments caused by trauma, tumor growth, or degenerative disc disease. The fixation devices immobilize the injured bone segments to ensure the proper growth of new osseous tissue between the damaged segments. Within the last few decades, the use of fixation plates for treatment of spinal disorders, or for fusion of vertebrae has grown considerably. While early procedures using fixation plates were at the lower lumbar levels, spinal fixation plates have recently found applications in the instrumentation of the cervical spine.

Successful spinal instrumentation in the cervical spinal region is particularly difficult given the problems of safely accessing the instrumentation site. The upper cervical spine can be approached either anteriorly or posteriorly, depending upon the spinal disorder to be treated. Bone fixation devices for the cervical spinal region often include internal bracing and instrumentation to stabilize the spinal column to facilitate the efficient healing of the damaged area without deformity or instability, while minimizing any immobilization and post-operative care of the patient.

One such device is an osteosynthesis plate, more commonly referred to as a bone fixation plate, which can be used to immobilize adjacent skeletal parts such as bones. Typically, the fixation plate is a rigid metal or polymeric plate positioned to span bones or bone segments that require immobilization with respect to one another. The plate is fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed.

Such osteosynthesis plates have been used to immobilize a variety of bones, including vertebral bodies of the spine. These bone plate systems usually include a rigid bone plate having a plurality of screw openings. The openings are either holes or slots to allow for freedom of screw movement. The bone plate is placed against the damaged vertebral bodies and bone screws are used to secure the bone plate to the spine, usually with the bone screws being driven into the vertebral bodies. Exemplary osteosynthesis plates are described in U.S. Pat. No. 6,159,213 to Rogozinski; U.S. Pat. No. 6,017,345 to Richelsoph; U.S. Pat. No. 5,676,666 to Oxland et al.; U.S. Pat. No. 5,616,144 to Yapp et al.; U.S. Pat. No. 5,549,612 to Yapp et al.; U.S. Pat. No. 5,261,910 to Warden et al.; and U.S. Pat. No. 4,696,290 to Steffee, the disclosures of each of which are incorporated by reference herein. Despite the existence of these osteosynthesis bone plates, there remains a need for a bone plate system that can provide increased visualization of a surgical site to facilitate alignment and implantation of the bone plate. There is also a need for a bone plate system which enables convenient installation of a bone screw through a drill guide.

There is a need for a cervical plating system which minimizes the intrusion into the patient and reduces trauma to the surrounding soft tissue. Moreover, a system is required that allows for easy access to drill and tap the cervical vertebrae with little room for error in positioning the fixation screw.

Even as cervical spine instrumentation techniques can be improved, so can the manner of fixation of the plate to the affected vertebral levels. For example, one cervical plating system sold by Synthes GmbH uses a cervical plate that accepts spinal screws at several locations disposed at the ends and in the middle of the plate. No matter which location is used, the screws are not capable of varying degrees of fixation between the vertebra and the plate. In addition, the Synthes device utilizes a locking screw which is threaded into the expansion head of the vertebral fixation screw to lock the screw into the plate. This procedure requires a locking screw for every fixation screw, thereby lengthening and complicating the procedure.

Therefore, there remains a need for a cervical plating system which provides for a wider range of fixations at the different vertebral levels. The need also extends to a plating system which minimizes the steps required to provide firm fixation of the spinal screws to the plate.

### SUMMARY

In one illustrative embodiment, the present invention includes anterior cervical plating systems which include an elongated anterior cervical plate having at least a pair of bone screw bores disposed near two opposite ends. The anterior cervical plate incorporates a curved plane transverse to the longitudinal axis to conform to the anterior surface of a vertebra and is also curved along the longitudinal axis to conform to the lordotic curvature between the vertebrae. Paired locking mechanisms are provided on the anterior cervical plate to engage bone screws by the heads of the bone screws, trapping the screws within the recesses of the anterior cervical plate, or interfacing with the neck portion of the bone screw under the anterior cervical plate. The locking mechanisms thus provide a rigid fixation of the bone screws to the anterior cervical plate with the heads of the bone screws flush or recessed below the upper curved surface of the anterior cervical plate. One or more through windows may be disposed in a middle bridge portion to provide a visualization pathway for a bone graph as well as an interface for a drill and tap guide assembly, which can be mounted on the anterior cervical plate to provide a firm foundation for accurately drilling and tapping screw holes into the vertebra to be instrumented by an assembly support engaged to the anterior cervical plate by way of a male positioning surface that interfaces with the through windows in the middle bridge portion, thus allowing a tap sleeve and drill guide to be supported by the assembly support, and thereby providing accurate positioning for a drill or tap.

Additional embodiments, examples, and advantages of cervical plating systems in accordance with the principles of the present invention will be apparent to those of ordinary skill in the art from the following specification.

### DESCRIPTION OF THE DRAWINGS

It will be appreciated by those of ordinary skill in the art that the elements depicted in the various drawings are not to scale, but are for illustrative purposes only. The nature of the present invention, as well as other embodiments of the present invention may be more clearly understood by reference to the following detailed description of the invention, to the appended claims, and to the several drawings attached hereto.

FIG. 1 is a bottom view of one illustrative embodiment of an anterior cervical plate in accordance with the principles of the present invention.

FIG. 2 is a top view of the anterior cervical plate of FIG. 1.

FIG. 3 is a cross-sectional side view, taken along line A1-A1 of FIG. 2, of the anterior cervical plate shown in FIGS. 1 and 2.

FIG. 4 is an end cross-sectional view, taken along line B1-B1 of FIG. 2, of the anterior cervical plate shown in FIGS. 1 through 3.

FIG. 5A is a bottom view of the anterior cervical plate of FIGS. 1 through 4, depicting the bone screw locking component in an unlocked position.

FIG. 5B is a bottom view of the anterior cervical plate of FIGS. 1 through 5A, depicting the bone screw locking component in a first locked position.

FIG. 5C is a bottom view of the anterior cervical plate of FIGS. 1 through 5B, depicting the bone screw locking component in a second locked position.

FIG. 6 is a cross-sectional side view of an assembled anterior cervical plate assembly including the anterior cervical plate of FIGS. 1 through 5C.

FIG. 7 is a bottom view of a second illustrative embodiment of an anterior cervical plate in accordance with the principles of the present invention.

FIG. 8 is a top view of the anterior cervical plate of FIG. 7.

FIG. 9 is a side cross-sectional view, taken along line A2-A2 of FIG. 8, of the anterior cervical plate shown in FIGS. 7 and 8.

FIG. 10 is an end cross-sectional view, taken along line B2-B2 of FIG. 8, of the anterior cervical plate shown in FIGS. 7 through 9.

FIG. 11A is a top view of the anterior cervical plate of FIGS. 7 through 10 depicting the bone screw locking component in an unlocked position.

FIG. 11B is a top view of the anterior cervical plate of FIGS. 7 through 11A depicting the bone screw locking component in a locked position.

FIG. 12 is a cross-sectional side view of an assembled anterior cervical plate assembly anterior cervical plate of FIGS. 7 through 11B.

FIG. 13A is a top view of a bone screw for use in a system in accordance with the principals of the present invention.

FIG. 13B is a side view of the bone screw of FIG. 13A.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

Anterior cervical plating systems in accordance with the present invention include anterior cervical plates having a unique geometry which render such anterior cervical plates effective and convenient to install. In anterior spinal cervical plate applications, for example, these anterior cervical plates may provide for enhanced visibility of the vertebral bodies to which they are mounted. These anterior cervical plate systems may also facilitate ease of installation through a design which enables the use of a guide device which can maintain registration with a bone screw hole during screw installation. As a result, such systems facilitate anterior cervical plate alignment, as well as proper and efficient placement of bone screws.

Illustrative anterior cervical plating systems in accordance with the present invention typically include an elongated anterior cervical plate having at least a pair of bone screw bores disposed near opposite ends along the longitudinal axis of the anterior cervical plate, and may have one or more through windows disposed in a middle bridge portion of the anterior cervical plate to provide an interface for a drill and tap guide assembly which may also provide a visualization pathway for the bone graph as well. Such anterior cervical plates may incorporate a curved plane transverse to the longitudinal axis to conform to the anterior surface of a vertebra and are curved along the longitudinal axis to conform to the lordotic curvature between the vertebrae and may be provided in differing longitudinal lengths to facilitate different physiologies. These arcs define a multi-axial circular arc, which can extend over the anterior cervical plates' length and width. Such curves may eliminate or reduce the need to bend the anterior cervical plate at the surgical site during the instrumentation procedure.

A paired locking mechanism may be provided on the anterior cervical plate to retain the bone screws within the recesses of the anterior cervical plate. Such a locking mechanism can provide a rigid fixation of the bone screws to the anterior cervical plate, while the heads of the bone screws reside in bone screw bores and are flush or recessed below the upper curved surface of the anterior cervical plate. This provides an advantage over prior systems that include components projecting above the anterior cervical plate, which can lead to irritation and trauma of the surrounding tissue.

In one illustrative embodiment, the paired locking mechanism engages the heads of bone screws, thereby locking the head of the bone screws to the anterior cervical plate. In such embodiments, the locking mechanism engages the anterior cervical plate at its upper or lower surface to clamp the bone screw heads within recesses in the anterior cervical plate. The bone screws and the locking mechanisms may be configured so that these components are flush with or below the upper surface of the anterior cervical plate.

In a second illustrative embodiment, the paired locking mechanism interfaces with a neck portion of a bone screw under the anterior cervical plate after the bone screws are inserted in the screw bores. In such an embodiment, the locking mechanism may be at least one locking plate movably disposed on the underside of the lower surface arc of the anterior cervical plate. Once a bone screw is installed, a locking plate may be slidably moved towards the neck of the bone screw, trapping the neck within the plate, thus providing a rigid fixation of the bone screw to the anterior cervical plate, while the head of the bone screw resides in a bone screw bore and is flush or recessed below the upper curved surface of the anterior cervical plate.

In some illustrative embodiments, the paired locking mechanism may include at least two sliding sleeves that are movably disposed over and around the upper surface arc of the anterior cervical plate. Once bone screws are installed, each sliding sleeve may be slidably moved over the heads of a pair of bone screws installed in a pair of screw bores, retaining the screws therein and providing a rigid fixation of the bone screws to the anterior cervical plate with the heads of the bone screws residing in the bone screw bores and flush or recessed below the upper curved surface of the anterior cervical plate.

Anterior cervical plate systems in accordance with the principles of the present invention, may include a single level anterior cervical plate (spanning between only two vertebral bodies) having at least two sets of bone screw bores, the sets disposed near opposite ends along the longitudinal axis of the anterior cervical plate. In other embodiments, the anterior cervical plate can be a multilevel anterior cervical plate spanning between three or more vertebral bodies and having sets of bone screw bores disposed near opposite ends along the longitudinal axis of the anterior cervical plate and one or more additional sets of bone screw bores disposed at intervals along the anterior cervical plate longitudinal axis length, the additional sets of screw bores disposed at each additional construct level.

Each set of screw bores may include two bores spaced apart from each other along a centerline of the anterior cervical plate. The central axes of a pair of screw bores may diverge relative to one another, such that a pair of bone screws placed therein will diverge relative to each other at a desired angle, which may be about twenty-two degrees.

The screw bores of the anterior cervical plates of the present invention may be countersunk to allow the enlarged heads of inserted bone screws to reside therein. This allows for the intrusion and trauma to tissue surrounding the implantation site to be minimized. This may also achieve a smooth outer contour for the instrumentation upon implantation.

A drill and tap guide assembly may be mounted on the anterior cervical plate to provide a firm foundation for accurately drilling and tapping screw holes into the vertebra to be instrumented. Such a drill and tap guide assembly includes an assembly support which is engaged to the anterior cervical plate by way of a male positioning surface that interfaces with the through windows disposed in the middle bridge portion of the anterior cervical plate. A tap sleeve and drill guide can then be supported by the assembly support, thereby providing accurate positioning for a drill or tap.

Such a guide device may have a handle portion, a guide barrel, and at least one connection structure extending from a distal end of the guide barrel. The guide device may be adapted to interface with the through window or windows in the middle bridge portion of the anterior cervical plate through the connection structure. The connection structure may engage an upper curved surface of the anterior cervical plate. For example, the connection structure of the guide device can include one or more tabs having a shape corresponding to a curved outer side surface of the anterior cervical plate which are spaced or configured to interface with the through windows in the middle bridge portion of the anterior cervical plate. Similarly, the drill guide can include two guide barrels and be adapted to register with two bone screw bores.

In addition to anterior cervical plates, systems for anterior fixation of the cervical spine in accordance with present invention may include several bone screws, one for each of the screw bores of an accompanying anterior cervical plate. Each of the bone screws may include an elongated shank with bone threads and an enlarged head for a corresponding screw bore at an upper surface of the anterior cervical plate when the shank extends there-through.

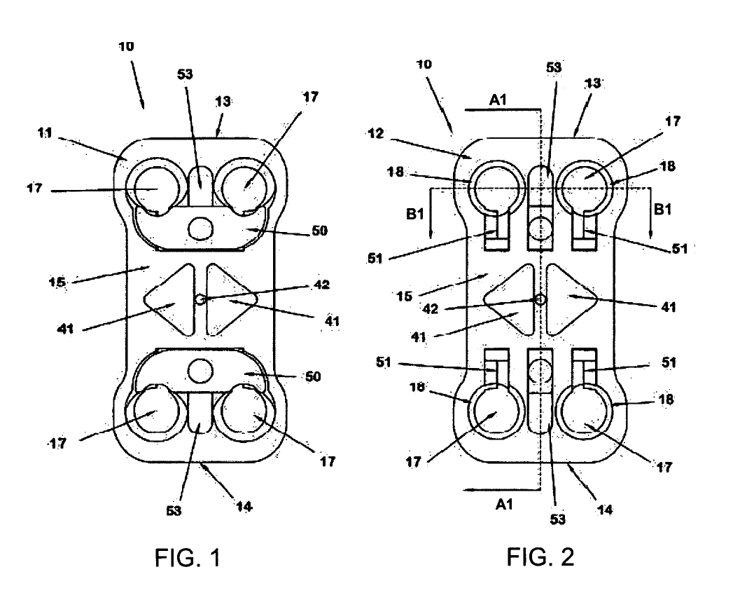

Referring now to FIGS. 1, 2, 3, and 4, a first embodiment of an elongated anterior cervical plate 10, in accordance with the principles of the present invention is depicted. Anterior cervical plate 10 includes a lower surface 11, and an opposite upper surface 12, as well as a relatively wider first end 13 and opposite wider second end 14 which are spanned by a narrower bridge portion 15.

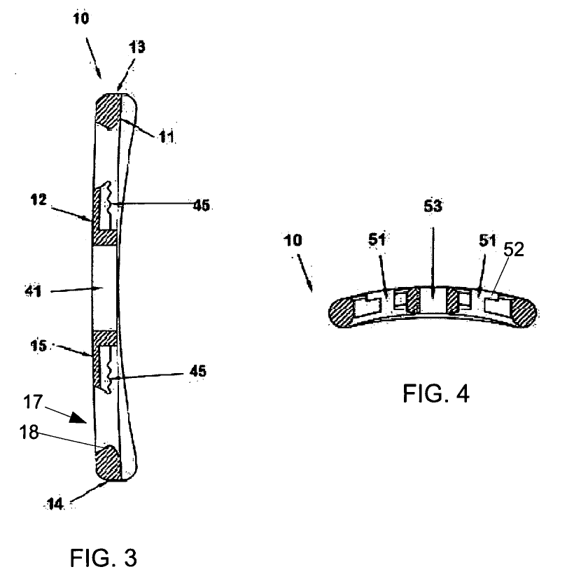

A number of screw bores 17 are disposed in the anterior cervical plate 10. In the depicted embodiment, a set of two screw bores is oriented at each of the first end 13 and the second end 14. Thus, in the depicted embodiment, four screw bores 17 are included such that when bone screws are mounted in the anterior cervical plate 10 through each of these bores 17 a solid quadrilateral fixation to the instrumented vertebrae is obtained. Each of the bores 17 includes a recessed rim 18, which may have a spherically-shaped surface, defined from the upper surface 12 of the anterior cervical plate, which are may be most clearly depicted in FIG. 3.

Anterior cervical plate 10 includes at least two through windows 41 in the middle bridge portion 15 to provide a visualization pathway for the bone graph as well as an interface for a drill and tap guide assembly. As depicted in FIGS. 1 and 2, such through windows 41 may be configured as bilaterally symmetrical triangles with bases near a midline of bridge 15, but it will be appreciated that other configurations may be used. A threaded hole 42, shown in FIGS. 1 and 2, is centrally located on the curved plane transverse to the longitudinal axis to provide an interface for drill and tap guide assembly. It will further be appreciated that in other embodiments, the through windows 41 may be omitted and only the threaded hole 42 provided, or a single centrally-located through window may be provided.

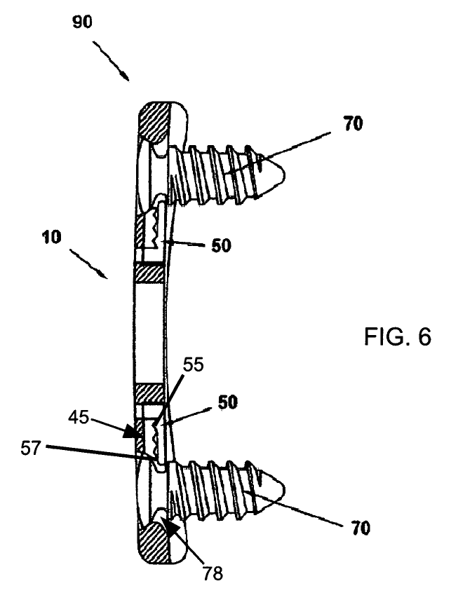

Raised serrations 45 (FIG. 6) are provided as a stepped interface between the anterior cervical plate 10 and two locking plates 50 which are movably disposed to the underside of the lower surface 11 arc of the anterior cervical plate 10. Raised serrations 45 allow the two locking plates 50 to be securely restrained throughout the range of movement at a desired position within that range of movement.

The relationship of a locking plate 50 and anterior cervical plate 10 interface 51 is depicted in FIGS. 2 and 4. Locking plate 50 is held to the anterior cervical plate 10 through a tongue and groove configuration, as shown by groove 52 of interface 51. Locking plate 50 can be accessed through the top surface 12 by way of a through slot 53, which allows locking member 50 to be slid into various locking positions.

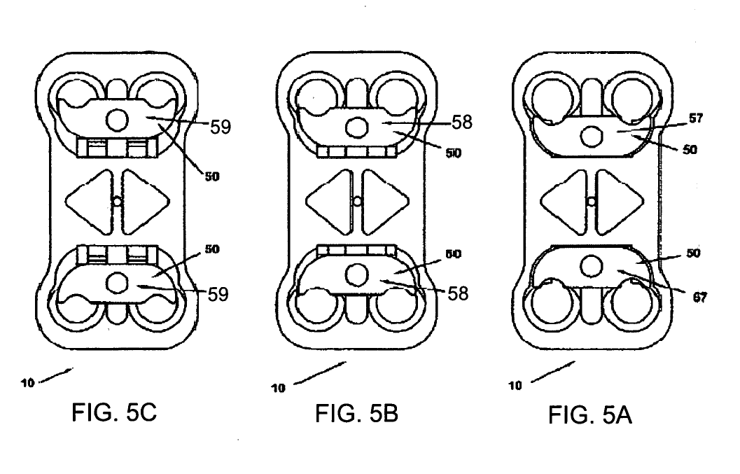

FIG. 5A depicts anterior cervical plate 10 with its two locking plates 50 that are movably disposed to the underside the lower surface arc, with the two locking plates 50 in the un-locked position 57. FIG. 5B, depicts the two locking plates 50 in a first locked position 58 and FIG. 5C depicts the two locking plates 50 in the second locked position 59.

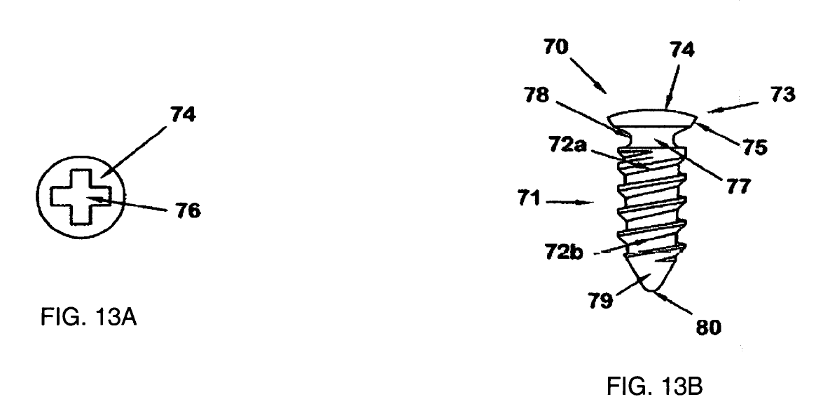

FIG. 6 depicts anterior cervical plate 10, with its two locking plates 50 in the second locked position 59, after insertion of bone screws 70 into the screw bores 17. Bone screw 70 is discussed in additional detail in connection with FIGS. 13A and 13B. As depicted, the locking plates 50 may be restrained in the locked position by the interaction of a projection 55, or ridge on an upper surface of plate 50 with the serrations 45. The curved leading edge 57 of each plate 50 may engage a neck portion 78 of the bone screw 70, retaining the screw 70 within the recess.

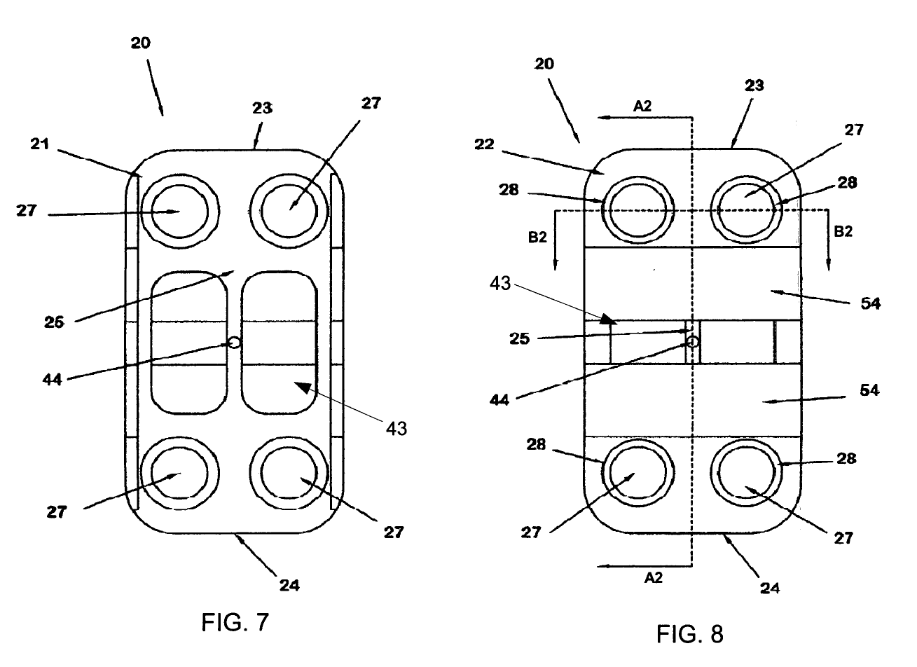

Referring now to FIGS. 7, 8, 9, and 10, a second embodiment of an elongated anterior cervical plate 20 is depicted. Anterior cervical plate 20 includes a lower surface 21, and an opposite upper surface 22, as well as a first end 23 and an opposite second end 24 spanned by a bridge portion 25, which may have a similar width to the end portions of the plate 20.

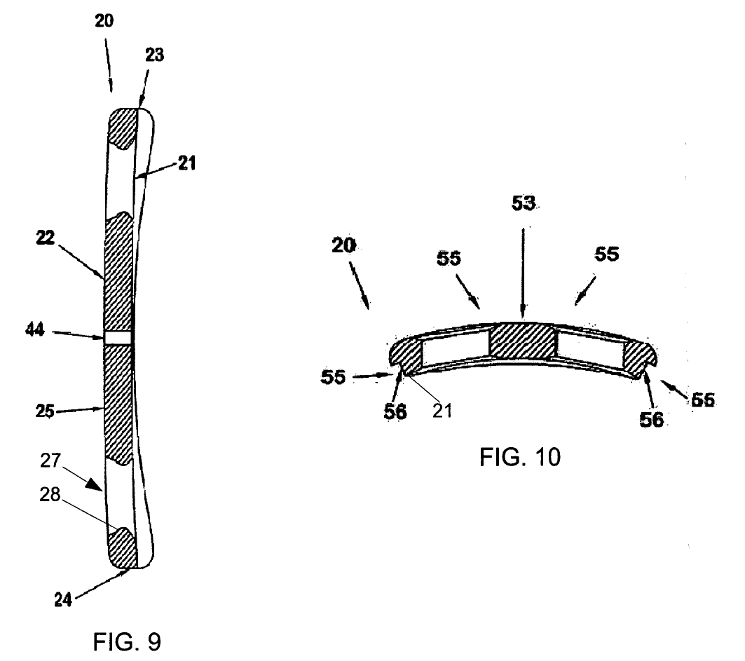

Anterior cervical plate 20 may include a number of screw bores 27 defined therein. As with the anterior cervical plate 10, a set of two screw bores 27 may be disposed at a position towards each of first end 23 and second end 24, such that when fixation screws are mounted in each of the screw bores 27, the anterior cervical plate may be provided a solid quadrilateral fixation to the instrumented vertebrae. Each of the bores 27 may include a recessed rim 28, which may have a spherically-shaped cross-section, defined from the upper surface 22 of the anterior cervical plate, as shown more clearly in FIG. 9.

Anterior cervical plate 20 of the present embodiment may also include at least two through windows 43 in middle bridge portion 25 to provide a visualization pathway for the bone graph as well as an interface for a drill and tap guide assembly. As depicted in FIGS. 7 and 8, the two through windows 43 may be configured as bilaterally symmetrical rounded rectangles, but it will be appreciated that other configurations may be used. A threaded hole 44 is centrally located on the curved plane transverse to the longitudinal axis to provide an interface for drill and tap guide assembly.

The interface 55 between sliding locking sleeve 54 and anterior cervical plate 20 can be most clearly discerned in FIGS. 7 and 10. Each locking sleeve 54 may be formed as a sliding plate held to the anterior cervical plate 20 through a tongue and groove configuration 56 along the longitudinal edge of the anterior cervical plate 20. This structure is repeated along both longitudinal edges for both of the locking sleeves 54. Thus, in the depicted embodiment, two sliding locking sleeves 54 are movably disposed over and around the upper surface arc of the anterior cervical plate.

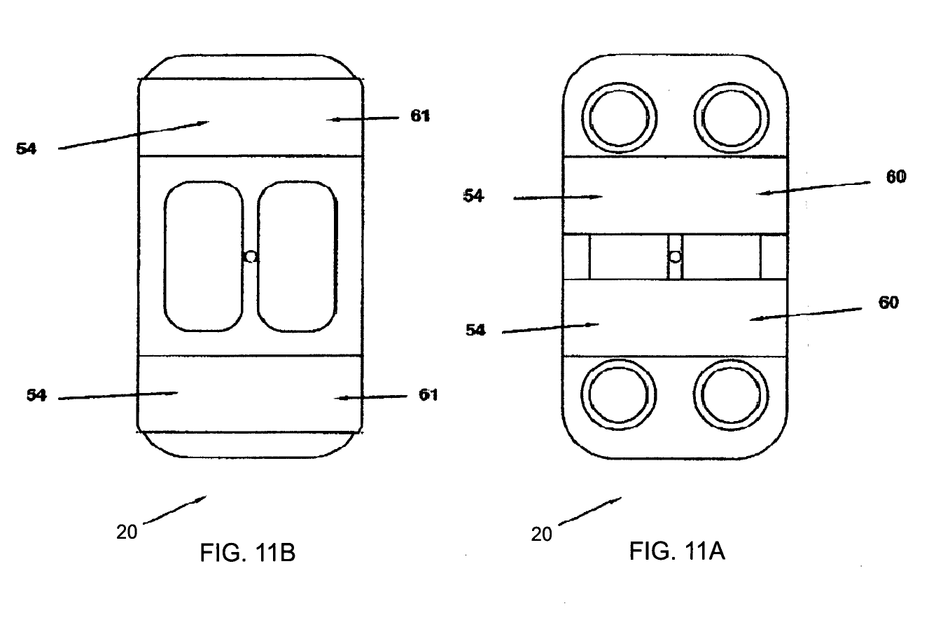

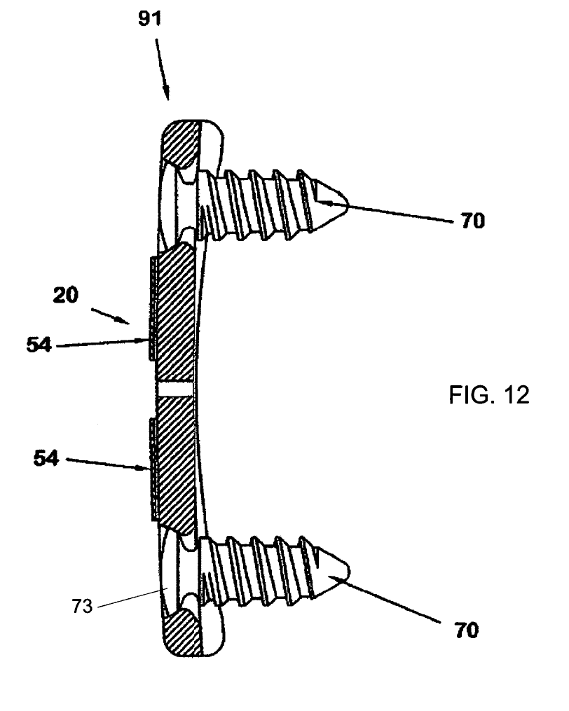

FIGS. 11A and 11B illustrates the anterior cervical plate 20 with the two sliding sleeves 54 movably disposed over and around the upper surface arc of the anterior cervical plate, in the un-locked position 60 (FIG. 11A) and in the locked position 61 (FIG. 11B) to retain screw heads 73 (FIG. 12) within the screw bores 27. Each sliding sleeve 54 may be retained in the locked position 61 by friction fit or by a separate locking mechanism.

FIG. 12 depicts a cross-sectional view of anterior cervical plate 20, with its two locking sleeves 54 in the unlocked position 60, after insertion of bone screws 70 into the screw bores 27. Bone screws 70 are discussed in additional detail in connection with FIGS. 13A and 13B. As depicted, the lower surface of the screw heads rests upon the spherical recess 28 of the screw bores 27 in the anterior cervical plate 20, and will be retained therein upon movement of the locking sleeve 54 into the locked position 61.

FIGS. 13A and 13B, illustrate one embedment of an illustrative bone screw 70 for use with systems in accordance with the present invention. Bone screw 70 includes an elongated tapered (as shown) or straight shank 71 having a lower threaded portion 72b and an upper smooth portion 72a. Adjacent the smooth portion 72a is an enlarged head 73 of the bone screw 70. Head 73 includes a spherical upper surface 74 and an opposite spherically cut lower surface 75. Lower surface 75 is curved to match the curvature of the spherical recess 18 of the screw bores 17 in the anterior cervical plate 10 or of the spherical recess 28 of the screw bores 27 in the anterior cervical plate 20. The neck 77 illustrates a concaved surface 78 about the longitudinal axis of the bone screw 70. The tip 79 of the bone screw 70 may define a sharp or blunted point 80, as tip 79 may be either a self-tapping or non self-tapping tip 79. The upper surface 74 defines a driving tool recess 76 formed therein. The driving tool recess 76 is adapted to engage a standard driving tool, such as a Phillips Head or similar configuration.

The drill and tap guide assembly may be a second plate that attaches above and/or over the upper surface of the anterior cervical plate 10 or 20 by attachment in the through windows 41 or 43 or threaded hole 42 or 44. One or more arms or fingers may extend through or around the anterior cervical plate 10 or 20 from the guide assembly to contact the vertebrae to which attachment is desired. The guide assembly will have guide holes aligned with the bore holes 17 or 27 of the anterior cervical plate 10 or 20. This allows a tap or bit inserted into the guide holes to pass through the bore holes 17 or 27 and contact the vertebrae to prepare for the emplacement of bone screws 70, while preventing contact with the anterior cervical plate 10 or 20 (as at recessed spherical edge 18 or 28) and without requiring removal of the anterior cervical plate 10 or 20.

The components of the anterior cervical plate system in accordance with the principles of the present invention can be made of any sturdy biocompatible material suitable for an orthopedic application. Suitable materials may include titanium, stainless steel, and alloys containing the same.

While the present invention has been shown and described in terms of preferred embodiments thereof, it will be understood that this invention is not limited to any particular embodiment and that changes and modifications may be made without departing from the true spirit and scope of the invention as defined and desired to be protected.

1. An elongated anterior cervical plate for anterior fixation of the spine, comprising:
an upper surface;
a lower surface;
a longitudinal axis running from a first end to an opposite second,
a curved plane transverse to the longitudinal axis to conform to an anterior surface of a vertebra;
a second curved plane parallel to the longitudinal axis to conform to the lordotic curvature between vertebrae;
at least a first pair of bone screw bores disposed at a point nearer the first end of the anterior cervical plate relative to a midpoint of the anterior cervical plate along the longitudinal axis;
a second pair of bone screw bores disposed at a point nearer the second end of the anterior cervical plate relative to a midpoint of the anterior cervical plate along the longitudinal axis;
at least a first locking mechanism disposed to retain a bone screw, when the bone screw is inserted into one of the at least a first pair of bone screw bores, to retain the bone screws within the bone screw bore with a head of the bone screw flush or recessed below the upper surface of the anterior cervical plate, the at least a first locking mechanism comprising a locking plate that is movably disposed on the underside of the lower surface and which retains the bone screw by interfacing with a neck portion of the bone screw by inserting an edge of the locking plate into a recess on the neck of the bone screw; and
at least a second locking mechanism disposed to retain a bone screw, when the bone screw is inserted into one of the second pair of bone screw bores, to retain the bone screws within the bone screw bore with a head of the bone screw flush or recessed below the upper surface of the anterior cervical plate.

2. The elongated anterior cervical plate of claim 1, wherein the at least a first locking mechanism retains two bone screws, one inserted into each of the at least a first pair of bone screw bores.

3-4. (canceled)

5. The elongated anterior cervical plate of claim 1, wherein the at least a second locking mechanism comprises a second locking plate that is movably disposed to on the underside of the lower surface and which retains the bone screw by interfacing with a neck portion of the bone screw by inserting an edge of the locking plate into a recess on the neck of the bone screw.

6. The elongated anterior cervical plate of claim 1, further comprising a stepped interface between the anterior cervical plate and the locking plate and a projection on the upper surface of the locking plate that interacts with the stepped interface to retain the locking plate in a desired position.

7. The elongated anterior cervical plate of claim 1, wherein centerlines of the at least a first pair of bone screw bores diverge relative to each other at an angle of about twenty-two degrees.

8. The elongated anterior cervical plate of claim 1, wherein each of the at least a first pair of screw bores includes a recessed rim with a spherically-shaped surface.

9. The elongated anterior cervical plate of claim 1, further comprising at least one through window in a middle bridge portion of the anterior cervical plate.

10. The elongated anterior cervical plate of claim 9, wherein the at least one through window in a middle bridge portion of the anterior cervical plate comprises a threaded hole.

11. The elongated anterior cervical plate of claim 9, wherein the at least one through window in a middle bridge portion of the anterior cervical plate comprises two symmetrical windows separated by a middle bridge portion.

12. A system for anterior fixation of the spine, comprising:
an elongated anterior cervical plate comprising
a longitudinal axis running from a first end to an opposite second end,
a curved plane transverse to the longitudinal axis to conform to an anterior surface of a vertebra;
a second curved plane parallel to the longitudinal axis to conform to the lordotic curvature between vertebrae;
at least a first pair of bone screw bores disposed at a point nearer the first end of the anterior cervical plate relative to a midpoint of the anterior cervical plate along the longitudinal axis;
a second pair of bone screw bores disposed at a point nearer the second end of the anterior cervical plate relative to a midpoint of the anterior cervical plate along the longitudinal axis;
at least a first locking mechanism disposed to retain a bone screw, when the bone screw is inserted into one of the at least a first pair of bone screw bores, to retain the bone screws within the bone screw bore with a head of the bone screw flush or recessed below the upper surface of the anterior cervical plate, the at least a first locking mechanism comprising a locking plate that is movably disposed at the underside of a lower surface of the cervical plate which retains the bone screw by interfacing with a neck portion of the bone screw by inserting an edge of the locking plate into a recess on the neck of the bone screw;
at least a second locking mechanism disposed to retain a bone screw, when the bone screw is inserted into one of the second pair of bone screw bores, to retain the bone screws within the bone screw bore with a head of the bone screw flush or recessed below the upper surface of the anterior cervical plate; and
at least a first bone screw for insertion into a bone screw bore of the anterior cervical plate.

13-14. (canceled)

15. The system for anterior fixation of the spine of claim 12, wherein the at least a second locking mechanism comprises a locking plate that is movably disposed at the underside of the lower surface of the cervical plate which retains the bone screw by interfacing with a neck portion of the bone screw by inserting an edge of the locking plate into a recess on the neck of the bone screw.

16. The system for anterior fixation of the spine of claim 12, wherein the at least a first bone screw for insertion into a bone screw bore of the anterior cervical plate comprises a threaded shank portion, an enlarged head, and a grooved neck disposed between the threaded shank and the enlarged head.

17. The system for anterior fixation of the spine of claim 12, further comprising a stepped interface between the anterior cervical plate and the locking plate and a projection on the upper surface of the locking plate that interacts with the stepped interface to retain the locking plate in a desired position.

18. The system for anterior fixation of the spine of claim 12, wherein centerlines of the at least a first pair of bone screw bores diverge relative to each other at an angle of about twenty-two degrees.

19. The system for anterior fixation of the spine of claim 12, wherein each of the at least a first pair of screw bores includes a recessed rim with a spherically-shaped surface.

20. The system for anterior fixation of the spine of claim 12, wherein the at least a first bone screw for insertion into a bone screw bore of the anterior cervical plate comprises an enlarged head with a spherical lower surface curved to match the curvature of the recessed rim of the at least a first pair of bone screw bores.

21. The system for anterior fixation of the spine of claim 12, further comprising at least one through window in a middle bridge portion of the anterior cervical plate.

22. The system for anterior fixation of the spine of claim 22, wherein the at least one through window in a middle bridge portion of the anterior cervical plate comprises two symmetrical windows separated by a middle bridge portion.

23. The system for anterior fixation of the spine of claim 12, wherein the at least a first bone screw for insertion into a bone screw bore of the anterior cervical plate comprises at least four bone screws, each incorporating an elongated shank having a lower threaded portion and an upper smooth portion, and an enlarged head.
